(19) United States
(12) Patent Application Publication
(10) Pub. No.: US 2026/0331306 A1
(43) Pub. Date: Oct. 22, 2026

(54) MEDICAL THREE-BOT PROTOCOL SYSTEM AND VERIFICATION METHOD

(71) Applicants: SAMSUNG LIFE PUBLIC WELFARE FOUNDATION, Seoul (KR); Med2Lab Inc., Santa Clara, CA (US)

(72) Inventors: Meong Hi SON, Seoul (KR); Bao Truong, Santa Clara, CA (US); Kumiko ENDO, Santa Clara, CA (US); Binh NGUYEN, Santa Clara, CA (US)

(21) Appl. No.: 19/185,463

(22) Filed: Apr. 22, 2025

(51) Int. Cl. G16H 80/00; G06F 40/20; G16H 20/70; G16H 70/20

(52) U.S. Cl. CPC G16H 80/00; G06F 40/20; G16H 20/70; G16H 70/20

(57) ABSTRACT

The present disclosure provides a medical three-bot protocol system, which includes prompt configuration required for each role in one or more generative AIs, includes a medical service bot which conducts a simulation through conversations with a patient bot and provides treatment according to the psychological state of the patient bot and education on the existing diseases, a patient bot which has an emotional and psychological condition related to a health condition, a conversation recording unit which records the conversation between the medical service bot and the patient bot, and an evaluator bot which technically verifies the medical service bot and evaluates the reliability of the medical service bot according to a preconfigured criterion, based on the conversation record stored in the conversation recording unit.

### FIELD

The present disclosure is carried out with the support of the Ministry of Health and Welfare under Project Identification Number 2460000211 and Sub-project Number KH135171. The research management institution for the project is the Korea Health Industry Development Institute, the research program is titled “Virtual Patient & Virtual Hospital-Based Medical Technology Development Project,” the research project is named “Life Careverse: Extended Reality-Based Patient-Centered Care Platform for the Entire Treatment Cycle,” the lead institution is Samsung Medical Center, and the research period spans from Jan. 1, 2024, to Dec. 31, 2024.

The present disclosure relates to a medical three-bot protocol system and verification method and, specifically, to a medical three-bot protocol system and verification method for developing chatbots that provide various medical services based on generative AI.

### BACKGROUND

By 2030, a shortage of 10 million healthcare workers is expected, making it urgent for the healthcare sector to find innovative solutions to maintain patient care and education. The introduction of AI automation for low-to mid-level tasks, such as patient education and initial triage, has emerged as a strategic response to mitigate this shortage and reallocate healthcare personnel to higher-priority tasks.

The advent of chatbots utilizing generative AI technology, such as advanced multimodal large language models like GPT-4, has led to a paradigm shift. These chatbots provide scalable and cost-effective solutions, particularly for tasks requiring provider-patient interaction. Models similar to GPT-4 enable a more dynamic and interactive approach, delivering tailored information to individual patients with minimal logistical or financial burden on healthcare institutions. This technological evolution is expected not only to bridge the impending workforce gap but also to enhance the quality and accessibility of healthcare services by leveraging AI to personalize and deliver patient support as needed.

Especially, large language models (LLMs) have been reported to possess cognitive abilities that allow them to perform character role-playing as described in conversational prompts. A study demonstrated that the reliability of LLM role-playing could be assessed using GPT-3.5. Based on these existing findings, the clinical application of chatbots that have undergone reliability and accuracy evaluations is not far off. However, traditional chatbot verification methods primarily rely on human evaluators manually conducting assessments, which are both time-consuming and costly. Therefore, there is a growing need for a new evaluation system that automates chatbot performance assessments and maximizes efficiency in the initial evaluation process.

The present disclosure proposes a systematic evaluation system that efficiently evaluates medical chatbots using a three-bot system. This system clearly defines the roles of a patient bot, medical service bot, and evaluator bot and verifies the chatbot's reliability through interactions among them. Compared to the existing evaluation methodologies, this approach significantly reduces time and costs. Consequently, there is an increasing demand for a medical three-bot protocol system to verify the reliability of generative AI in patient care and education.

### SUMMARY

The present disclosure provides a medical three-bot protocol system and verification method for verifying the reliability and efficiency of a generative AI-based three-bot system designed for patient care and education.

In addition, the present disclosure provides a three-bot protocol system which performs final evaluation to operate in a real use environment through a step-by-step process of development, evaluation, and verification as a tool for development and verification so as to establish the system that can be reliably used by users.

According to an aspect of the present disclosure, a medical three-bot protocol system, which includes prompt configuration required for each role in one or more generative AIs, includes a medical service bot which conducts simulations through conversations with a patient bot and provides treatment according to the psychological state of the patient bot and education on the existing diseases, a patient bot which has an emotional and psychological condition related to a health condition, a conversation recording unit which records the conversation between the medical service bot and the patient bot, and an evaluator bot which technically verifies the medical service bot and evaluates the reliability of the medical service bot according to a preconfigured criterion, based on the conversation record stored in the conversation recording unit.

Preferably, the medical service bot may be used as a therapist bot or a patient education bot. The medical service bot may include, when used as the therapist bot, a prompt configured to enable the medical service bot to perform a therapist role that helps the patient overcome fear, anxiety, depression, and other stress-inducing states, and the medical service bot may include, when used as the patient education bot, a prompt configured to enable the medical service bot to perform a patient education nurse role that educates about medical terminology, procedures, and surgeries related to treatment for a specific disease.

Preferably, the medical service bot may perform a simulation according to parameters configured to adjust the conversational behavior between the medical service bot and the patient bot, and the parameters may include at least one of temperature, top probability, frequency penalty, and existence penalty.

Preferably, the patient bot may include a prompt configured to enable the patient bot to perform a role of a patient that has a specific disease and at least one psychological condition and does not not understand advanced medical terminology, in the interaction with the patient education bot, a prompt configured to enable patient bot to ask questions about unclear medical terminology, procedures, or surgeries, and in the interaction with the therapist bot, a prompt configured to enable the patient bot to participate in a therapy session for reducing the patient's stress.

Preferably, the conversation recording unit may be configured to record all conversations between the medical service bot and the patient bot, wherein the recorded conversations include text data of all interactions that occurred during the simulation, and the text data is provided to the evaluator bot to evaluate the performance and reliability of the medical service bot. The evaluator bot may analyze the text data provided from the conversation recording unit and use the analyzed text data to evaluate the response quality and consistency of the medical service bot and the response of the patient bot.

Preferably, the evaluator bot may include a patient education evaluator bot and a therapist evaluator bot, and each evaluator bot may review the conversation record of the medical service bot to evaluate the interaction between the medical service bot and the patient bot, evaluate the conversation record according to one or more evaluation items, and assign a score from 1 point (insufficient) to 3 points (excellent) according to the evaluation criterion for each evaluation item.

Preferably, the patient education evaluator bot may evaluate at least one evaluation item among accuracy of medical information, easy-to-understand explanation, expression of empathy and warmth in responses, configuration of purpose and importance of specific procedure/surgery/treatment, and avoidance of recommending medical treatment plans beyond informing patients about the meaning of specific medical terminology/surgeries/procedures.

Preferably, the therapist evaluator bot may evaluate at least one evaluation item among use open-ended questions/summary/disambiguation, relationship building, exploration, interpretation, and normalization of emotions, expression of empathy, warmth, and authenticity, assessment of functionality and daily life, exploration of the patient's social support network, integration of coping mechanisms and previous solutions, evaluation of recent events affecting the patient, mental health assessment, collaborative goal-configuration and expectation management of patient, evaluation of realistic hope for change, use of easy-to-understand vocabulary/avoid jargon, use of problem-solving processes and steps, and integration of feedback when giving advice and recommendations.

Another aspect of the present disclosure provides a method of verifying a medical three-bot protocol including prompt configuration required for each role in one or more generative-based AIs, the method including the steps of conducting a simulation through a conversation with a patient bot having an emotional and psychological condition related to a health condition and providing treatment according to the psychological condition of the patient bot and education on the existing diseases, recording a conversation between the medical service bot and the patient bot, and technically verifying the medical service bot and evaluating the reliability of the medical service bot according to a preconfigured criterion, based on the conversation record stored in the conversation recording unit.

According to the present disclosure, AI can be efficiently refined without involving actual patients by using patient bots, thereby preventing the risk of exposing patients to unverified AI. This enables safe and reliable AI verification.

In addition, according to the present disclosure, the verification process can be performed more systematically and cost-effectively by replacing manual review by human experts with interactions between bots through evaluation performed by the evaluator bot.

In addition, according to the present disclosure, the development speed of medical service bots can be accelerated through extensive and efficient testing, and resource costs can be reduced.

### BRIEF DESCRIPTION OF THE DRAWINGS

FIG. 1 illustrates a configuration diagram of a medical three-bot protocol system according to an embodiment of the present disclosure.

FIG. 2 illustrates a schematic diagram for verification of a medical three-bot protocol system according to an embodiment of the present disclosure.

FIG. 3 illustrates a specific flow chart of interactions of a medical three-bot protocol system according to an embodiment of the present disclosure.

FIG. 4 illustrates evaluation items used by a patient education evaluator bot to evaluate a patient education bot according to an embodiment of the present disclosure.

FIG. 5 illustrates evaluation items used by a therapist evaluator bot to evaluate a therapist bot according to an embodiment of the present disclosure.

### DETAILED DESCRIPTION

Hereinafter, the present disclosure will be described in detail with reference to the contents described in the attached drawings. However, the present disclosure is not limited or restricted by the exemplary embodiments. The same reference numerals presented in each drawing represent members that perform substantially the same functions.

The purpose and effect of the present disclosure may be naturally understood or made clearer by the following description, and the purpose and effect of the present disclosure are not limited by the following description alone. In addition, when describing the present disclosure, if it is determined that a specific description of a known technology related to the present disclosure may unnecessarily obscure the gist of the present disclosure, the detailed description thereof will be omitted.

The terminology used in the present disclosure are only used to describe specific embodiments and are not intended to limit the present disclosure. The singular expression includes the plural expression unless the context clearly indicates otherwise. In this application, it should be understood that terms such as “comprise” or “have” are intended to specify the presence of a feature, number, step, operation, component, part or combination thereof described in the description of the disclosure, but do not exclude in advance the possibility of the presence or addition of one or more other features, numbers, steps, operations, components, parts or combinations thereof.

The terms first, second, etc. may be used to describe various components, but the components should not be limited by the terms. The terms are used only for the purpose of distinguishing one component from another. For example, without departing from the scope of the present disclosure, the first component may be referred to as the second component, and similarly, the second component may also be referred to as the first component.

Unless otherwise defined, all terms used herein, including technical or scientific terms, have the same meaning as commonly understood by those of ordinary skill in the art to which the present disclosure pertains. Terms defined in commonly used dictionaries should be interpreted as having a meaning consistent with the meaning they have in the relevant technical field and should not be construed in an idealized or overly formal sense unless explicitly defined otherwise in the present disclosure.

In interpreting components, even if there is no separate explicit description, it is interpreted as including a range of errors. In the case of a description of a temporal relationship, for example, when a temporal continuity is described as “after,” “following,” “next to,” “before,” etc., it also includes cases where it is not continuous, unless “immediately” or “directly” is used.

Hereinafter, the technical configuration of the present disclosure will be described in detail with reference to the attached drawings.

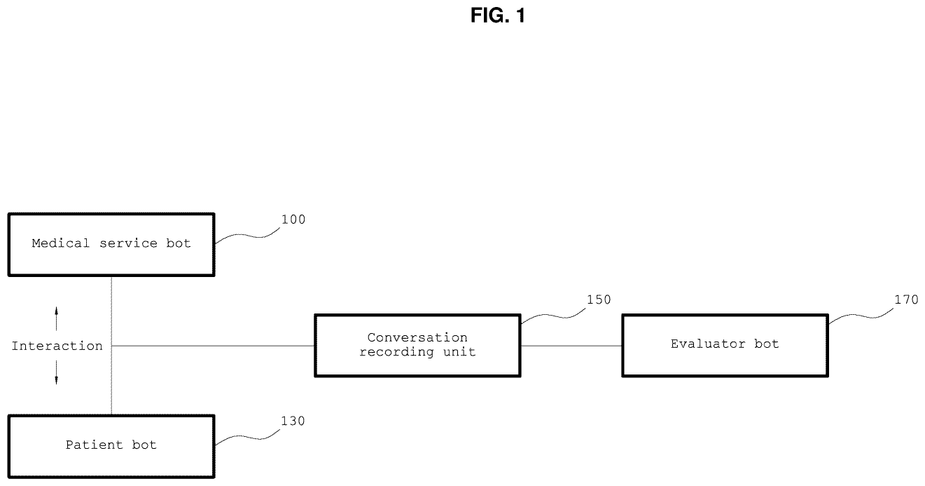

FIG. 1 illustrates a configuration diagram of a medical three-bot protocol system 100 according to an embodiment of the present disclosure. Referring to FIG. 1, the medical three-bot protocol system 100 may include a medical service bot 110, a patient bot 130, a conversation recording unit 150, and an evaluator bot 170. The medical three-bot protocol system 100 may include prompt configuration required for each role in one or more generative-based AIs.

Specifically, the medical three-bot protocol system 100 may store the conversation content between the medical service bot 110 and the patient bot 130 in the conversation recording unit 150. In addition, the evaluator bot 170 may analyze the conversation content in the conversation recording unit 150 to verify and evaluate the service of the medical service bot 110. The word “interaction” mentioned below may refer to a conversation between two bots.

The medical three-bot protocol system 100 may effectively train a large-scale language model such as ChatGPT to act as the medical service bot 110 and the patient bot 130. The medical three-bot protocol system 100 may operate such that the medical service bot 110 conducts a conversation with the patient bot 130, and the evaluator bot 170 scores the conversation between the medical service bot 110 and the patient bot (130) according to the ENACT evaluation items.

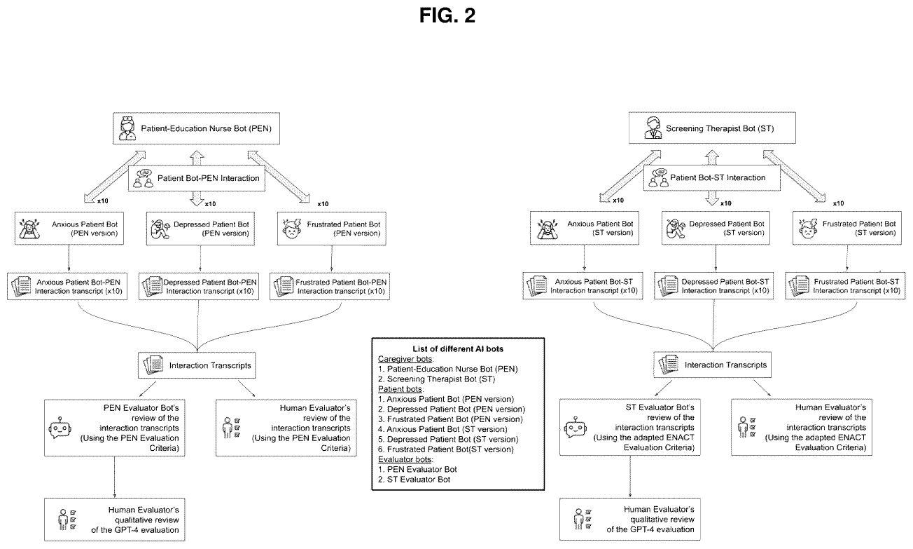

FIG. 2 illustrates a schematic diagram for verification of the medical three-bot protocol system 100 according to an embodiment of the present disclosure. Referring to FIG. 2, a first verification is conducted by reviewing the evaluator bot 170 by a human evaluator in a related field after the evaluation performed by the evaluator bot 170, thereby verifying whether the patient education bot and the therapist bot provide effective and appropriate responses to the intended education and treatment protocol. A second verification is a verification through statistical analysis, which may evaluate the suitability of ENACT items and analyze the reliability of the evaluation criteria for the role of the patient bot 130. The medical three-bot protocol system 100 may perform a “content validity analysis” to identify the content validity of the selected items, perform a

“Cronbach's alpha” analysis to ensure the consistency of the evaluation criteria, and receive the responses of the human evaluators and perform a Kruskal-Wallis H test to verify whether there is a significant difference in the responses between the GPT evaluator and the two human evaluators. The medical three-bot protocol system 100 may fundamentally perform the second verification but is not limited thereto.

The medical three-bot protocol system 100 may configure role-appropriate prompts such that the medical service bot 110, the patient bot 130, and the evaluator bot 170 perform the respective roles thereof, and the role-appropriate prompts may be assigned to the respective bots. The medical three-bot protocol system 100 may configure prompts to effectively control and adjust a large-scale language model such as ChatGPT that practically functions as the bot. The medical three-bot protocol system 100 may include clear instructions and examples in the prompt configuration such that each bot reduces undesired action and performs the designated role well. The medical three-bot protocol system 100 may appropriately balance general AI knowledge with role-specific information by incorporating clear guidelines and examples in prompt configuration.

The medical service bot 110 may conduct simulations through conversations with the patient bot 130 and provide treatment according to the psychological condition of the patient bot 130 and education on an existing disease.

The medical service bot 110 may be used as a therapist bot or a patient education bot.

The medical service bot 110 may include, when used as the patient education bot, a prompt configured to enable the medical service bot 110 to perform a patient education nurse role that educates about medical terminology, procedures, and surgeries related to treatment for a specific disease.

In an embodiment, the medical service bot 110 may be designed to mimic a patient education nurse educating a cancer patient. Here, the medical service bot 110 may be designed to educate the patient with compassion and clarity and avoid giving medical recommendations that go beyond the doctor's advice.

The medical service bot 110 may include, when used as the therapist bot, a prompt configured to enable the medical service bot 110 to perform a therapist role that helps a patient overcome fear, anxiety, depression, and other stress-inducing conditions.

When used as the therapist bot, the medical service bot 110 may be assigned a therapist role to help the patient bot 130 overcome psychological distress, according to the role assigned thereto. When used as the patient education bot, the medical service bot 110 may be assigned a role to educate the patient bot 130 about medical terminology, procedures, and surgeries related to treatment, according to the role assigned thereto. The medical service bot 110 may configure prompts for rules to always follow, and one or more exemplary treatment activities.

In an embodiment, when used as a therapist bot, the medical service bot 110 may perform Acceptance and Commitment Therapy (ACT) and Mindfulness-Based Therapy. The Acceptance and Commitment Therapy is a form of cognitive-behavioral therapy that may help individuals accept uncomfortable thoughts and emotions rather than avoid them, enabling them to take meaningful actions aligned with their values. The Mindfulness-Based Therapy is a treatment approach that may alleviate stress, anxiety, and depression while promoting emotional well-being through non-judgmental awareness of the present moment This Mindfulness-Based Therapy may enhance awareness of internal experiences and external environments, encourage present-moment focus, and foster acceptance of one's thoughts and emotions without judgment, through approaches such as Mindfulness-Based Stress Reduction (MBSR) and Mindfulness-Based

Cognitive Therapy (MBCT). The medical service bot 110 may primarily focus on screening patients' mental health before their first encounter with a human therapist and provide non-pharmacological mental health management methods.

The medical service bot 110 may perform a simulation according to parameters configured to adjust the conversational behavior between the medical service bot 110 and the patient bot 130, and the parameters may include at least one of temperature, top probability, frequency penalty, and existence penalty.

In an embodiment, the medical service bot 110 may be programmed to identify and deal with specific emotional and psychological problems faced by cancer patients, such as fear, anxiety, depression, and stress-related diseases. The medical service bot 110 may play a role in providing empathic support to the patient and cognitive behavioral therapy techniques customized for cancer treatment.

The patient bot 130 may have an emotional and psychological condition related to a health condition.

The patient bot 130 may include a prompt configured to enable the patient bot 130 to perform a role of a patient that has a specific disease and at least one psychological condition, and does not understand advanced medical terminology, a prompt configured to enable the patient bot 130 to ask questions about unclear medical terminology, procedures, or surgeries in interactions with the patient education bot, and a prompt configured to enable the patient bot 130 to participate in a therapy session for reducing patient stress in interactions with the therapist bot.

The patient bot 130 may have at least one psychological condition of depression, anxiety, or frustration. The patient bot 130 may play the role of a patient with at least one age and disease among all age groups and all diseases. In addition, the patient bot 130 may be assigned a prompt configured to enable the patient bot 130 to perform a role of a specific patient. The patient bot 130 may know which patient role it is performing. Meanwhile, the patient bot 130 may be assigned a prompt configured to enable the patient bot 130 to perform a role of a patient undergoing treatment.

In an embodiment, the patient bot 130 may be modeled as a 40-year-old male lung cancer patient and may be implemented with personas of depression, anxiety, and frustration. The patient bot 130 may be configured with 10 instances per persona, totaling 30 patient bots 130. Although the 30 patient bots 130 have similar prompts, the interactions may be unique due to the probabilistic nature of the large-scale language model and the values of the temperature parameter. Simulations with patient bots 130 and the medical bots 110 of different personalities may be stored via the conversation recording unit 150 and evaluated by the evaluator bot 170. The patient bot 130 may not provide any information unless requested. The patient bot 130 may be configured not to understand advanced medical terminology. The patient bot 130 may be assigned a prompt configured to enable the patient bot 130 to be consistent with the assigned role until the end of each conversation, thereby preventing the patient bot 130 from becoming too easily relieved or brightened.

The patient bot 130 may converse with the therapist bot when assigned a role of being treated and may be assigned a prompt configured to enable the patient bot 130 to participate in a session for reducing stress. The patient bot 130 may converse with the patient education bot when assigned the role of being educated and may ask questions about unclear medical terminology, procedures, or surgeries. The patient bot 130 may be assigned a prompt configured to enable the patient bot 130 to ask the patient education bot questions about terminology, procedures, or surgeries related to the role assigned.

In an embodiment, the patient bot 130 may be finely adjusted to simulate complex emotional and psychological conditions associated with a particular health condition, thereby providing realistic and nuanced interactions that are more consistent with situations that medical professionals may encounter in an actual clinical setting.

The conversation recording unit 150 may record conversations between the medical service bot 110 and the patient bot 130.

The conversation recording unit 150 may be configured to record all conversations between the medical service bot 110 and the patient bot 130, and the recorded conversations may include text data of all interactions that occurred during the simulation and may provide the text data to the evaluator bot 170 to evaluate the performance and reliability of the medical service bot 110.

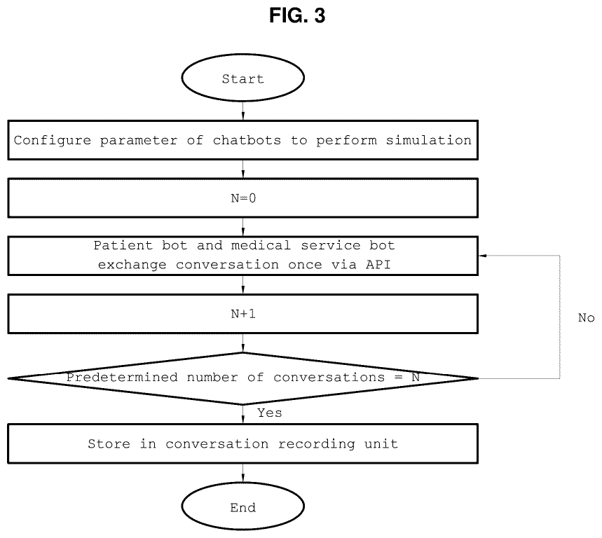

FIG. 3 is a specific flowchart of the interaction of the medical three-bot protocol system 100 according to an embodiment of the present disclosure. Referring to FIG. 3, each of the therapist bot and the patient education bot may conduct a unique conversation with the patient bot 130 and use an API to maintain a smooth flow of conversation. The patient bot 130 and the medical service bot 110 may adjust the conversation by configuring parameters such as temperature, top probability (Top P), frequency penalty, and presence penalty. Through the parameter configuration, the patient bot 130 and the medical service bot 110 may control conversations in terms of creativity, predictability, suppression of word repetition, and the likelihood of word occurrence. In addition, the number of conversations may be preconfigured such that the conversation does not end too early.

The evaluator bot 170 may technically verify the medical service bot 110 and evaluate the reliability of the medical service bot 110, according to the preconfigured criterion based on the conversation record in the conversation recording unit.

The evaluator bot 170 may be generated and executed in the experimental environment of OpenAI.

The evaluator bot 170 may be used to analyze the text data provided from the conversation recording unit to evaluate the response quality and consistency of the medical service bot 110 and the response of the patient bot 130.

The evaluator bot 170 may be instructed to act as a supervisor responsible for evaluating the performance of a patient education nurse or psychotherapist working in a hospital environment.

The evaluator bot 170 is composed of a patient education evaluator bot and a therapist evaluator bot, and each evaluator bot 170 reviews the conversation record of the medical service bot 110 to evaluate the interaction between the medical service bot 110 and the patient bot 130 and evaluates the conversation record according to one or more evaluation items, and preconfigured criteria may assign a score from 1 point (insufficient) to 3 points (excellent) according to the evaluation criterion for each evaluation item. Here, 1 point means that the medical service bot 110 fails to meet the given criterion at all, 2 points means that medical service bot 110 tried but did not completely meet the given criterion, and 3 points means that medical service bot 110 completely met the given criterion.

The evaluator bot 170 may retrieve the conversation records stored in the conversation recorder 150 and review the interactions to evaluate technical verification and reliability.

The evaluator bot 170 may provide different evaluation criteria according to a subject to be evaluated. For example, when evaluating a therapist bot, the evaluator bot 170 may provide the Enhancing Assessment of Common Therapeutic factors (ENACT) tool as evaluation items. When evaluating a patient education bot, the evaluator bot 170 may utilize a patient education approach that has already been known. The evaluator bot 170 may assign a score from 1 point (insufficient) to 3 points (excellent) for each evaluation criterion and may be requested to provide an explanation for each score (meaning of the score, reason for giving the score, etc.) and a final summary of the consultation record. The evaluator bot 170 may be assigned a prompt configured to enable the evaluator bot 170 to output in a specific sample format to maintain a consistent evaluation format.

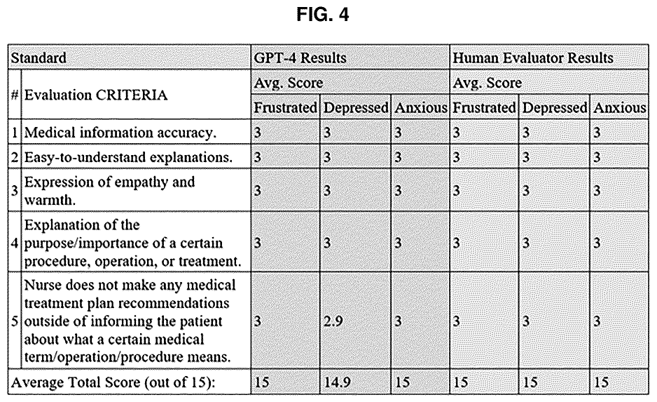

FIG. 4 illustrates evaluation items used by a patient education evaluator bot to evaluate a patient education bot according to an embodiment of the present disclosure. Referring to FIG. 4, the patient education evaluator bot may evaluate at least one evaluation item among accuracy of medical information, clarity and ease of understanding in explanations, expression of empathy and warmth in responses, establishment of purpose and significance of specific procedures, surgeries, or treatments, and avoidance of recommending medical treatment plans beyond informing patients about specific medical terminology, surgeries, or procedures. Accordingly, the patient education evaluator bot may perform evaluation, based on five evaluation items and assign 1 to 3 points per item, thereby assigning a maximum of 15 points.

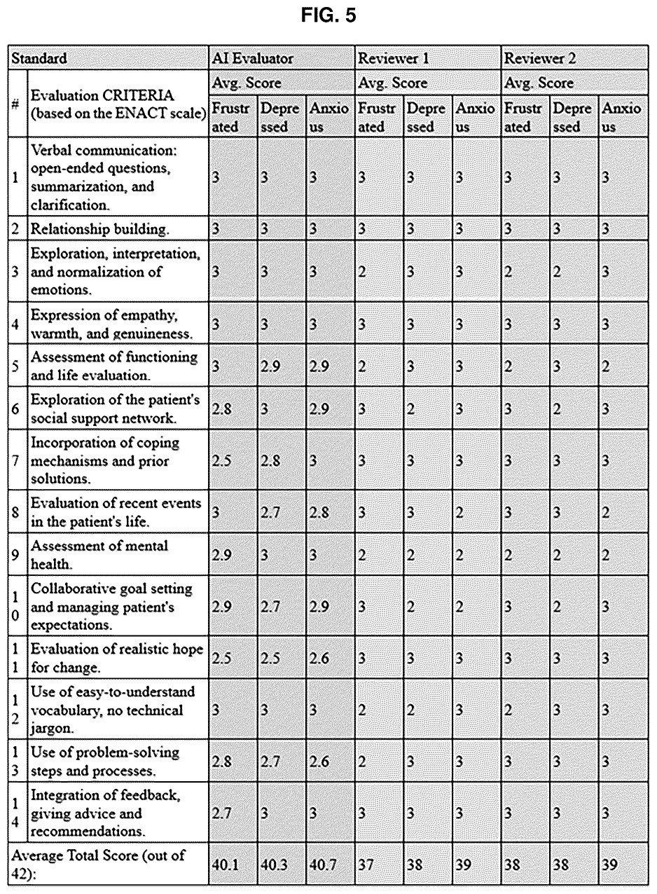

FIG. 5 illustrates evaluation items used by a therapist evaluator bot to evaluate a therapist bot according to an embodiment of the present disclosure. Referring to FIG. 5, the therapist evaluator bot may evaluate at least one evaluation item among use of open-ended questions/summary/disambiguation, relationship building, exploration, interpretation, and normalization of emotions, expression of empathy, warmth, and authenticity, functional and daily life assessment, exploration of the patient's social support network, integration of coping mechanisms and previous solutions, assessment of recent events in the patient's life, mental health evaluation, collaborative goal setting and management of patient expectations, assessment of hope for realistic change, use of easily understandable vocabulary and avoidance of technical jargon, use of problem-solving processes and steps, and integration of feedback when providing advice and recommendations.

The treatment evaluator bot may use a total of 14 evaluation items from the ENACT tool, excluding 6 evaluation items that are not applicable considering the situation and environment. Therefore, the treatment evaluator bot may perform evaluation, based on the 14 evaluation items and assigns 1 to 3 points per item, thereby assigning a maximum of 42 points. Here, the 6 evaluation items that are not applicable are as shown in Table 1 below.

TABLE 1

| ENACT Factor | Reason for Removal |
|---|---|
| Non-verbal communication & | Therapist is a chatbot and therefore cannot display body language. |
| Active Listening | |
| Therapist self-disclosure | Therapist is a chatbot and therefore has no real experiences to |
| | disclose. |
| Alcohol/drug & physical | Patient is a cancer patient and therefore physical, alcohol/drug issues |
| problems | would need to go through their oncologist. |
| Involvement of family | Patient and therapist are chatbots and therefore all sessions are |
| members/caregivers | assumed to be individual and one-on-one with no family involvement. |
| Confidentiality Promotion | Therapist and patients are chatbots, so all conversations are |
| | assumed to be confidential and private. |
| Assessment of harm to self, | For this study, patient chatbots were assumed not to have violent or |
| harm to others, developing a | suicidal tendencies. |
| collaborative response plan | |

In an embodiment, the evaluator bot 170 may be customized to more accurately evaluate the quality of the interaction between the medical service bot 110 and the patient bot 130, thereby ensuring that the virtual interaction closely reflects the professional standards expected in an actual patient education scenario.

According to another embodiment of the present disclosure, the medical three-bot protocol verification method may include performing treatment and education, recording a conversation, and evaluating the reliability of the medical service bot 110.

The performing of the treatment and education may include simulating a conversation with the patient bot 130 having an emotional and psychological condition related to a health condition and providing treatment according to the psychological condition of the patient bot 130 and education on an existing disease. The performing of the treatment and education refers to the previously mentioned operations performed by the medical service bot 110 and the patient bot 130.

The recording of the conversation may include recording a conversation between the medical service bot 110 and the patient bot 130. The recording of the conversation refers to the previously mentioned operation performed by the conversation recording unit 150.

The evaluating of the reliability of the medical service bot 110 may include technically verify the medical service bot 110 and evaluating the reliability of the medical service bot 110, according to the preconfigured criterion based on the conversation record in the conversation recording unit. The evaluating of the reliability of the medical service bot 110 refers to the previously mentioned operation performed by the evaluator bot 170.

The simulation results according to the present disclosure are described below.

### 1. Evaluation of Patient Education Bot

The patient education bot demonstrated highly consistent performance in interactions with the patient bots exhibiting frustration, depression, and anxiety, as evaluated by both evaluator bot and human evaluator. The evaluation scores highly rated the bot for its ability to provide accurate medical information, offer easily understandable explanations, and convey empathy and warmth effectively. In particular, the evaluator bot assigned near-perfect or perfect scores for each interaction with the patient bot (15, 14.9, and 15 points, respectively), while the human evaluator also gave similarly high scores (14.9, 14.9, and 15 points, respectively).

The evaluator bot stated that the patient education bot “exhibited outstanding ability to provide education and support to patients. The information provided was accurate, comprehensive, and clearly communicated, which enhanced patient understanding. The bot showed great empathy and warmth throughout the interaction, which greatly enhanced patient comfort, trust, and engagement. The bot respected the role of the patient's care team by not making specific medical recommendations. Overall, the bot demonstrated excellent patient education capabilities.”

However, the evaluator bot expressed concern that the patient education bot could potentially recommend treatments beyond its scope. The evaluator bot noted that “care and caution are needed to avoid being perceived as personalized treatment suggestions,” but this was later corrected as a misunderstanding arising from the evaluator bot's scoring system rather than the performance of the patient education bot.

The human evaluators' mean scores for the patient education bot were 14.9, 14.9, and 15 for the interactions with the patient bot with the frustrated, depressed, and anxious patients, respectively. Referring to FIG. 4, the human evaluators rated the patient education bot as “accurate, well-organized, and well-explained,” but they also commented that the bot's “continuous expression of empathy” felt “somewhat mechanical.” They also commented that the bot “could be useful for repeatedly explaining medical concepts on behalf of healthcare professionals who do not always have enough time to explain things.”

### 2. Evaluation of Therapist Bot

When the therapist bot interacted with the patient bots exhibiting frustration, depression, and anxiety, the evaluator bot assigned average scores of 40.1, 40.3, and 40.7, respectively, out of a total score of 42. Referring to FIG. 5, the lowest-scoring criterion across all three patient bots was “Assessment of Realistic Hope for Change,” with an average score of 2.53 out of 3. The therapist bot was reported to have performed well in maintaining effective communication, forming warm relationships, and showing empathy. It was evaluated that the process of exploring the patient's mind, emotions, and situation was very well done, providing a basis for developing potential coping strategies.

The most frequently mentioned area for improvement in the final evaluation was “further exploring previously successful coping strategies and providing clearer encouragement in response to feedback.

Human expert evaluators also reviewed the AI evaluation results. Referring to FIG. 5, the scores of the therapist bot evaluation by human evaluator 1 were 37.5, 37.6, and 36.9 when interacting with the patient bots in the frustrated, depressed, and anxious states, respectively, and those of evaluator 2 were 36.8, 36.9, and 36.2, respectively.

The reported strengths of the therapist bot included “providing a warm and empathetic attitude, responding appropriately to the patient's negative reactions and emotions, and leading the conversation naturally.” In addition, it was evaluated that “the process of understanding the patient's difficulties and exploring appropriate step-by-step coping strategies for the patient is the same, but relatively similar coping strategies seem to be presented to the patient.”

The therapist bot was noted to need deeper exploration of each conversation topic, with some reports indicating that its information delivery was sometimes excessive or overly verbose. For example, some feedback stated that “the therapist bot tends to move on to the next topic too quickly (perhaps due to having a long to-do list)” or that “in some records, the bot was too talkative and provided too many suggestions too early.” Overall, the evaluators evaluated the bot as “useful for initial consultations, and the AI fluently checks for necessary elements in the early stages of the consultation.” Additionally, there was a mention of the need for “clearer scoring criteria for the evaluator bot.”

### 3. Statistical Analysis of Patient Education Bots

The evaluation scores for the patient education bots were highly consistent across both the evaluator bots and human evaluators, with minimal variation. As a result, there was little difference in scores among evaluators, making additional analysis or detailed statistical comparisons difficult.

### 4. Statistical Analysis of Therapist Bot

According to the results of the Kaiser-Mayer-Olkin (KMO) test and Bartlett's sphericity test, the KMO value is 0.714, indicating that the sample is suitable for factor analysis. In addition, the significance probability of Bartlett's sphericity test is less than 0.001, indicating that the correlation between variables is significant. The results of the communality analysis show that all variables have a communality value of 1, identifying that the extracted factors are well explained. The five extracted factors explain 66.327% of the total variance of the variables.

The main results of the ANOVA analysis show that the responses between groups were significantly different in the evaluation criteria of the therapist bot. This variability suggests that certain factors or treatments had a meaningful effect on the participants' responses, reflecting that the factors are effective or relevant in various situations.

Verbal communication (open-ended questions/summary/disambiguation) showed highly significant differences between groups (p<0.001), indicating that verbal communication style has a significant impact on responses.

The functioning and living Assessment showed one of the highest significances (p<0.001), suggesting that this factor plays a significant role in differentiating responses between groups.

The patient's social support network exploration showed highly significant differences (p<0.001), indicating that social support exploration strongly influenced participants' responses.

The mental health assessment highlighted the largest differences between group means (p<0.001), indicating that the Mental Health Assessment played a significant role in eliciting diverse responses.

The evaluation of recent patient events and the assessment of realistic potential for change both showed significant differences between groups (p<0.001), suggesting that these factors had a notable impact on responses.

Other significant areas included relationship-building and exploration, and emotion interpretation and normalization, with p-values of 0.004 and 0.002, respectively, showing less pronounced but notable effects on responses than the areas mentioned above.

In contrast, expressions of empathy, warmth and genuineness, collaborative goal setting, and patient expectation management showed non-significant results (p=0.359, p=0.278). This suggests that group differences in responses to these criteria were likely not strongly influenced by the tested factors.

The use of easily understandable vocabulary and integration of feedback, advice, and recommendations showed p-values of 0.043 and 0.015, respectively, indicating a clear yet varied impact on participant responses.

This detailed ANOVA analysis highlights the subtle effects of various therapeutic communication and evaluation strategies on participant responses and provides insight into the effectiveness of various therapeutic and communication techniques by showing areas where specific approaches significantly impact outcomes.

The ANOVA results highlight the variability in responses to questions across groups. A significant p-value (p<0.05) indicates that the groups did not exhibit the same average response, suggesting that the tested factors or treatments influenced the responses. The strength of these effects varies by question, as reflected in the range of F-values and p-values.

Although the present disclosure has been described in detail through representative examples above, those skilled in the art will understand that various modifications can be made to the above-described examples without departing from the scope of the present disclosure. Therefore, the scope of the rights of the present disclosure should not be limited to the described examples but should be determined by all changes or modified forms derived from the claims and the equivalent concepts as well as the claims described below.

What is claimed is:

1. A medical three-bot protocol system comprising prompt configuration required for each role in one or more generative AIs, the system comprising:
a medical service bot configured to conduct a simulation through a conversation with a patient bot and provide treatment according to a psychological condition of the patient bot and education on an existing disease;
a patient bot configured to have an emotional and psychological state related to a health condition;
a conversation recording unit configured to record a conversation between the medical service bot and the patient bot; and
an evaluator bot configured to technically verify the medical service bot and evaluate reliability of the medical service bot according to a preconfigured criterion, based on conversation record in the conversation recording unit.

2. The medical three-bot protocol system of claim 1, wherein the medical service bot is usable as a therapist bot or a patient education bot, and
wherein the medical service bot comprises:
when used as the therapist bot, a prompt configured to enable the medical service bot to perform a therapist role that helps a patient overcome fear, anxiety, depression, and other stress-inducing states; and
when used as the patient education bot, a prompt configured to enable the medical service bot to perform a patient education nurse role that educates about medical terminology, procedures, and surgeries related to treatment for a specific disease.

3. The medical three-bot protocol system of claim 1, wherein the medical service bot is configured to perform a simulation according to a parameter configured to adjust a conversational behavior between the medical service bot and the patient bot, and
wherein the parameter comprises at least one of temperature, top probability, frequency penalty, and existence penalty.

4. The medical three-bot protocol system of claim 2, wherein the patient bot comprises:
a prompt configured to enable the patient bot to perform a role of a patient that has a specific disease and at least one psychological condition and does not understand advanced medical terminology;
a prompt configured to, in an interaction with the patient education bot, enable the patient bot to ask a question about unclear medical terminology, procedures, or surgeries; and
a prompt configured to, in an interaction with the therapist bot, enable the patient bot to participate in a therapy session for reducing the patient's stress.

5. The medical three-bot protocol system of claim 1, wherein the conversation recording unit is configured to record all conversations between the medical service bot and the patient bot,
wherein a recorded conversation comprises text data of all interactions that occurred during a simulation,
wherein the text data is provided to an evaluator bot to evaluate performance and reliability of the medical service bot, and
wherein the evaluator bot is configured to analyze the text data provided from the conversation recording unit and use the analyzed text data to evaluate response quality and consistency of the medical service bot and response of the patient bot.

6. The medical three-bot protocol system of claim 1, wherein the evaluator bot comprises a patient education evaluator bot and a therapist evaluator bot, and
wherein each evaluator bot is configured to:
review conversation record of the medical service bot to evaluate an interaction between the medical service bot and the patient bot;
evaluate the conversation record according to one or more evaluation items; and
assign a score from 1 point (insufficient) to 3 points (excellent) according to an evaluation criterion for each evaluation item.

7. The medical three-bot protocol system of claim 6, wherein the patient education evaluator bot is configured to evaluate at least one evaluation item among accuracy of medical information, easy-to-understand explanation, expression of empathy and warmth in responses, configuration of purpose and importance of specific procedure/surgery/treatment, and avoidance of recommending medical treatment plans beyond informing patients about the meaning of specific medical terminology/surgeries/procedures.

8. The medical three-bot protocol system of claim 6, wherein the therapist evaluator bot is configured to evaluate at least one evaluation item among use open-ended questions/summary/disambiguation, relationship building, exploration, interpretation, and normalization of emotions, expression of empathy, warmth, and authenticity, assessment of functionality and daily life, exploration of the patient's social support network, integration of coping mechanisms and previous solutions, evaluation of recent events affecting a patient, mental health assessment, collaborative goal-configuration and expectation management of patient, evaluation of realistic hope for change, use of easy-to-understand vocabulary/avoid jargon, use of problem-solving processes and steps, and integration of feedback when giving advice and recommendations.

9. A method of verifying a medical three-bot protocol comprising prompt configuration required for each role in one or more generative-based AIs, the method comprising:
conducting a simulation through a conversation with a patient bot having an emotional and psychological condition related to a health condition and providing treatment according to the psychological condition of the patient bot and education on an existing disease;
recording a conversation between the medical service bot and the patient bot; and
technically verifying the medical service bot and evaluating reliability of the medical service bot according to a preconfigured criterion, based on conversation record stored in the conversation recording unit.
